(19) United States
(12) Patent Application Publication
(10) Pub. No.: US 2010/0081922 A1
(43) Pub. Date: Apr. 1, 2010

(54) DIFFUSE OPTICAL TOMOGRAPHY

(75) Inventor: Alphonsus Tarcisius Jozef Maria Schipper, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/528,146

(22) PCT Filed: Feb. 27, 2008
(86) PCT No.: PCT/IB08/50698

§ 371 Date: Aug. 21, 2009

(30) Foreign Application Priority Data
Feb. 27, 2007 (EP) 07103114.0

(51) Int. Cl. A61B 6/00

(52) U.S. Cl. 600/425

(57) ABSTRACT

A diffuse optical tomography device comprises a light source for irradiating a mammalian body part, a holding device configured for holding the body part, a measuring device configured for measuring the light level in the holding device and the ambient light level and a controlling device configured for controlling the light source as a function of the measured light level in the holding device and the ambient light level. Such a device prevents light leakage into the eyes of a patient or an operator.

### FIELD OF THE INVENTION

The invention relates to a diffuse optical tomography device, more particularly to a controlling device included in the diffuse optical tomography device.

### BACKGROUND OF THE INVENTION

Optical Imaging is a promising new medical imaging modality. It is a non-harmful and inexpensive technique providing physiological information. An important application is breast cancer imaging, since breast cancer is very common and there is a clinical need for improved and earlier detection. It is the most frequently diagnosed cancer in women, and ranks second among cancer deaths in women. In many countries, screening programs are in place, aiming at early detection of breast cancer. These programs use X-ray mammography, which generates a large number of false positives and results in a large number of unnecessary biopsies. Other drawbacks of X-ray mammography are the use of ionizing radiation and uncomfortable breast compression. Optical fluorescence imaging holds the promise of imaging breast cancer with sufficient sensitivity and specificity. It can play a role in both the diagnostic process and therapy monitoring, because it is a women-friendly, non-harmful and relatively inexpensive technique.

Optical fluorescence imaging uses a fluorescence contrast agent and near-infrared light. After injection of the contrast agent, the breast is sequentially illuminated with laser light from all sides. Part of the light is absorbed in the breast by the contrast agent, which in turn lights up, producing fluorescence light with a different wavelength. The presence and position of a tumor can be detected by measuring this fluorescence light from all sides of the breast.

If laser light emitted through the light source leaks from the holding device, it could cause an eye injury either to a patient or to an operator. The laser light could burn a small portion of an eye retina, if the beam is accidentally aimed at the eyes of the patient or the operator. Such a beam sometimes could even cause blindness.

US20060013533 describes a method and apparatus for improving body safety during exposure to a monochromatic light source by diverging the monochromatic light with a highly durable diffuser. Eye safety is further enhanced by measuring the radiance of the divergent monochromatic light and issuing a warning as a result of a mishap if the radiance of the divergent monochromatic light is greater than a predetermined safe value, and if desired, generating a visible flash prior to the emission of a pulse of monochromatic light to induce an eye of a bystander to blink or to change its field of view in order to avoid staring at the monochromatic light. This method prevents a collimated beam to enter the eye by diverging it so that the energy density rapidly decreases with distance. Even the decreased energy density can be harmful to the eye and hence may require further countermeasures.

It would therefore be advantageous to have a system which prevents eye exposure without having any of the disadvantages described above.

Particular and preferred aspects of the invention are set out in the accompanying independent and dependent claims. Features of the dependent claims may be combined with features of the independent claims and with features of other dependent claims as appropriate and not merely as explicitly set out in the claims.

### SUMMARY OF THE INVENTION

According to a first aspect, the invention provides a diffuse optical tomography device comprising a light source for irradiating a mammalian body part, a holding device configured for holding the body part, a measuring device configured for measuring the light level in the holding device and the ambient light level, and a controlling device configured for controlling the light source as a function of the measured light level in the holding device and the ambient light level. The measuring device measures the light level in the holding device. The holding device is said to be dark when the amount of light in the holding device is below a threshold level. This threshold level is experimentally determined. The measuring device also checks for the presence of an ambient light level. The controlling device allows the light from the light source to enter the holding device only when the holding device is dark and ambient light is present. The low light level in the holding device indicates that no ambient light leaks into the holding device. It also means that no (laser) light leaks out of the holding device. In other words, the holding device is closed properly and is light leak proof. This ensures safety to a patient being examined or an operator operating the diffuse optical tomography device.

In the context of this invention, the light level in the holding device is measured. This may also be referred to as “darkness” or “amount of darkness” in the holding device. In effect what is measured is the very low light level in the holding device.

According to another embodiment of the invention, the measuring device further comprises a detector board. Preferably the device comprises a first series of optical fibers configured for sensing the light level in the holding device and for communicating to a detector board; a second series of optical fibers configured for transmitting the ambient light level to the detector board; and a third series of optical fibers configured for transmitting the scattered light from the holding device to the detector board. The fibers are preferably organized in rings in the holding device. Source and detector fibers are preferably interleaved per ring. The light level in the holding device is suitably checked through the first series of fibers originating from the topmost ring in the holding device. The ambient light level is checked through the second series of fibers originating from an ambient light source or from those fibers that do not have a fluorescence filter. The third series of fibers transmits the scattered light from the body part to the detector board. The first series of fibers and the second series of fibers ensure that the holding device is closed to prevent light leakage. The presence and position of a tumor is detected by measuring the light transmitted through the third series of fibers.

According to another embodiment of the invention, the controlling device comprises a shutter configured for preventing or allowing the light from the light source to enter into the holding device as a function of the measured light level in the holding device and the ambient light level. The shutter is not opened until the holding device is ensured to be closed and light leak proof. This enables safe operation of the diffuse optical tomography device.

According to yet another embodiment of the invention, a coupling fluid is disposed in the holding device to surround the body part, which coupling fluid has an optical characteristic substantially identical to an optical characteristic of the body part. The coupling fluid reduces the artifacts in a reconstructed image due to the boundary effect between the body part and the holding device. Moreover, if the coupling fluid is a liquid, a perfect match between the holding device and the shape of the body part can be obtained. The intensity difference in the image, due to different path lengths, between the light source and the measuring-controlling device can be equalized.

According to still another embodiment of the invention, the holding device includes a plurality of entrance openings for coupling the light generated by the light source into the holding device and a plurality of exit openings constructed to communicate scattered light to the measuring-controlling device, the scattered light being emitted from the body part in response to light from the light source. There are preferably 255 entrance and exit openings mounted in the wall of the holding device. The body part is sequentially illuminated using 255 optical fibers emerging from 255 entrance openings. Another 255 fibers emerging from 255 exit openings are used for detecting the scattered light for each illumination position. The fibers are preferably organized in rings. Source and detector fibers are interleaved per ring.

According to another embodiment of the invention, the light source is a laser light. By using a laser, the wavelength of the light to be generated can be adjusted in a range as small as about 10 nm around the center wavelength.

In another aspect according to the invention, a method for detecting optical leakage in an optical imaging system comprises the steps of:

placing a mammalian body part in a holding device of a diffuse optical tomography device;

irradiating the body part in the holding device by means of a light source;

measuring the light level present in the holding device by a measuring device;

measuring the ambient light level by the measuring device; and

controlling the light source based on the measured light level in the holding device and the ambient light level.

According to another embodiment of the invention, the light source is a laser light source.

According to yet another embodiment of the invention, a measuring device and a controlling device suitable for use in the tomography device are configured to measure the light level in the holding device and the ambient light level, and to control the light source of the tomography device as a function of the measured light level in the holding device and the measured ambient light level.

### DESCRIPTION OF THE FIGURES

These and other characteristics, features and advantages of the present invention will become apparent from the following detailed description, taken in conjunction with the accompanying drawings, which illustrate, by way of example, the principles of the invention. This description is given for the sake of example only, without limiting the scope of the invention. The reference figures quoted below refer to the attached drawings.

FIG. 1 shows the principle of optical fluorescence imaging;

FIG. 2 shows a schematic diagram of the safety concept for eye hazard;

FIG. 3 shows a cross sectional view of the holding device; and

FIG. 4 shows a top view of the holding device.

### DETAILED DESCRIPTION OF THE INVENTION

Particular and preferred aspects of the invention are set out in the accompanying independent and dependent claims. Features of the dependent claims may be combined with features of the independent claims and with features of other dependent claims as appropriate and not merely as explicitly set out in the claims.

The present invention will be described with respect to particular embodiments and with reference to certain drawings but is not limited thereto. Any reference signs in the claims shall not be construed as limiting the scope. The drawings described are only schematic and are non-limiting. In the drawings, the size of some of the elements may be exaggerated and not drawn to scale for illustrative purposes. Where the term “comprising” is used in the present description and claims, it does not exclude other elements or steps. Where the indefinite or definite article is used when referring to a singular noun e.g. “a” or “an”, “the”, this includes the plural of that noun unless something else is specifically stated.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

Moreover, the terms top, bottom, over, under and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

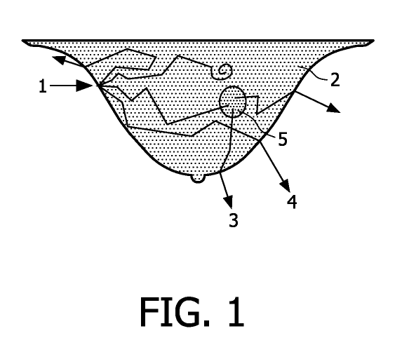

In FIG. 1, the mammalian body part 2 is sequentially illuminated with an incident light 1 from all sides. Part of the incident light 1 is absorbed in the body part 2 which in turn lights up, producing fluorescence light 3 of a different wavelength. The presence and position of a tumor 4 can be detected by measuring the fluorescence light 3 from all sides of the body part 2. Part of the incident light 1 is transmitted as transmitted light 4.

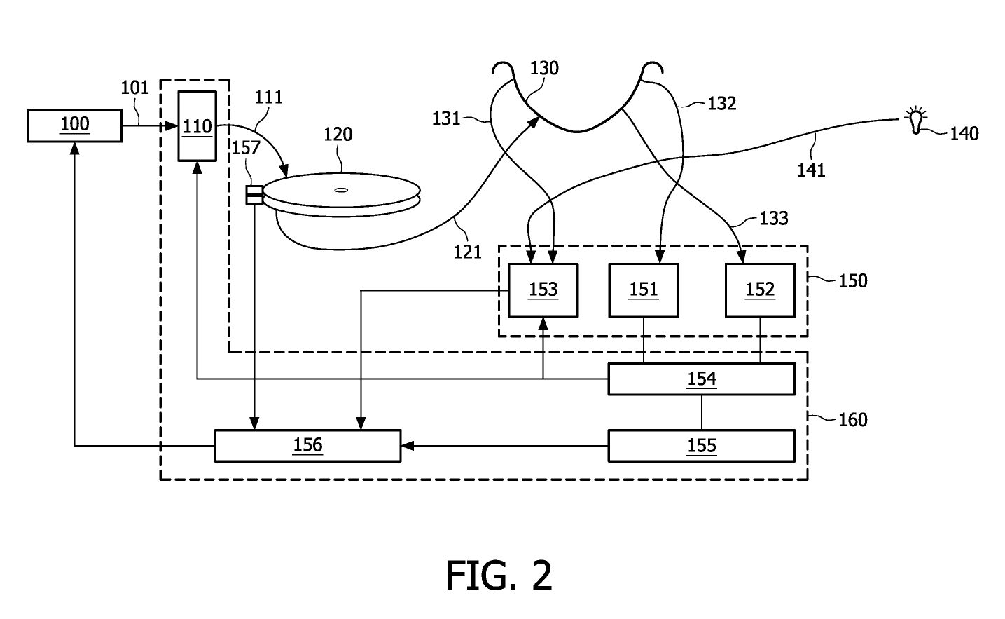

In FIG. 2, light from a laser light source 100 is fed through a fiber 101 to a shutter 110 and then to a fiber switch 120 through a fiber 111. The light further enters a holding device 130 configured to hold a mammalian body part (not shown). A measuring device 150 includes a plurality of detector boards (of which only two are shown i.e., 151 and 152) and a dedicated detector board 153. A controlling device 160 includes a detector controller 154, a computer 155 and a safety circuit 156. The safety circuit 156 includes a safety switch 157. A light source 140 provides ambient light. The fiber 101 transmits the light from the laser light source 100 to the shutter 110. The fiber 111 transmits the light from the shutter 110 to the fiber switch 120. The fiber 121 transmits the light from the fiber switch 120 to the holding device 130. The fiber 131 checks for the presence of ambient light in the holding device and communicates to the dedicated detector board 153. The fiber 141 transmits the ambient light from the light source 140 to the dedicated detector board 153. The fiber 132 senses the ambient light that enters the holding device and communicates to the detector board 151. The fiber 133 transmits the fluorescent and transmitted light from the holding device to the detector board 152. For convenience, only one fiber is shown in the Figure to represent a series of fibers.

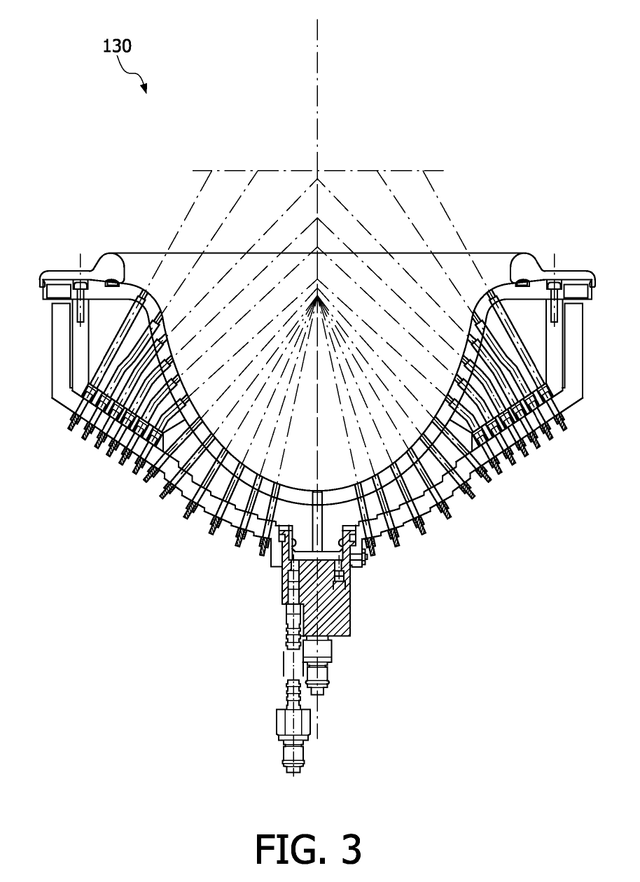

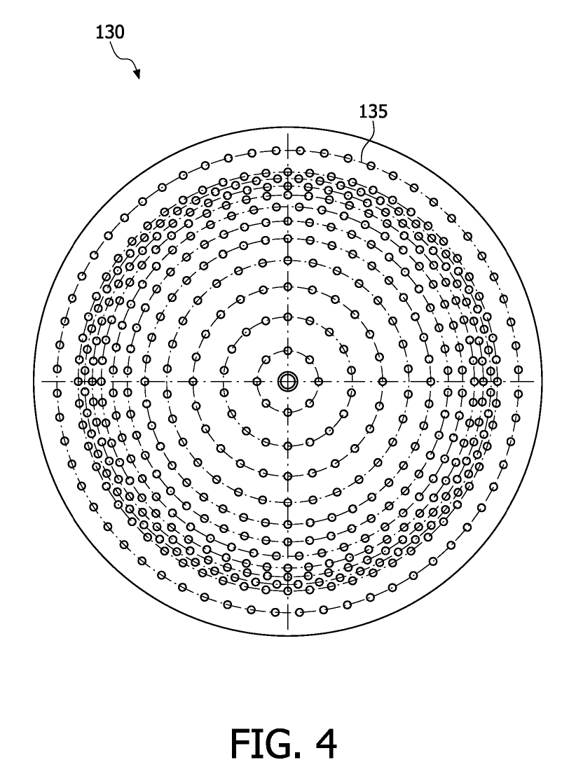

The cross sectional view of the holding device 130 is shown in FIG. 3. It also shows the entrance and exit openings of the holding device 130. On the other hand, FIG. 4 shows a top view of the holding device 130. The topmost ring is marked 135.

The light leakage detection has to be “single component failure safe”. This means that if one component fails, it should still be safe. Hence it is preferably built redundantly with two measures namely Countermeasure A and Countermeasure B.

For Countermeasure A, a dedicated detector Board 153 is used. Laser light from the light source 100 is fed through the fiber 101 to a shutter 110 and then to a fiber switch 120 through fiber 111. The dedicated detector board 153 checks for the presence of a light level in the holding device 130 through the fiber 131. The dedicated detector board also checks the ambient light outside the holding device 130 i.e., the light in the room emitted from the light source 140 through the fiber 141. Measures are taken to distinguish between the laser light and the ambient light when checking the light level in the holding device. This is done by temporarily disabling the light level check when the laser is pulsing into the holding device. If the measured ambient light level in the holding device 130 is below a threshold value, the holding device is said to be dark. The ambient light need not be very bright, as it may affect the quality of the image generated by the diffuse optical tomography device. The level of ambient light required is determined by the minimum amount of light required to measure the presence of ambient light in the holding device in case the holding device is slightly opened. The ambient light needed is around 100 lux. The light level in the holding device is determined as follows. The values generated by the detector board 153 when the holding device is fully closed and when the holding device is slightly opened (may be 5 to 10 mm) are noted down. When the holding device is slightly opened, it is ensured that a minimum amount of light in the room (ambient light) is present. The threshold value of the light level is in between these two values. It is measured in terms of arbitrary units. During the light level measurement, if the value generated by the detector is within the range of the above-mentioned values, the holding device is said to be dark. A fluorescence filter is generally present on the fibers to filter out light that is not infrared i.e., the light with wavelength below 780 nm. However, the fluorescence filter has been removed from fiber 131 to enable the light level measurement. The fibers in the holding device 130 are organized in rings. The fiber 131 originates from the top ring 135. If the holding device 130 is dark and the room has light, it means that room light is not leaking into the holding device, which further means that laser light from the holding device cannot leak out. Then the holding device 130 is assumed to be closed and laser light enters the holding device 130. This is signaled through the signal “HOLDING DEVICE_SAFE_A”.

HOLDING DEVICE_SAFE_A=(DARKNESS IN THE HOLDING DEVICE) AND (LIGHT IN THE ROOM)

The fiber switch 120 has a position in which the light is fed to the holding device 130 and a safe position (FS_SAFE) in which the laser light is not fed to the holding device 130. The safe position is detected by means of a safety switch 157 of which the output is fed to the safety circuit 156. In the safe position the fiber switch 120 serves as a laser light beam stopper. The measurement of light level is temporarily disabled while the shutter 110 is open. A signal from the shutter 110 is fed to the dedicated detector board 153. The dedicated detector board 153 also monitors the maximum time the shutter 110 remains opened through this signal. Ambient light from the light source 140 is checked through more than one fiber 141 from more than one position. This is to ensure that the ambient light is measured even when the operator accidentally covers one fiber. The room is considered lit if any fiber detects light. This is the Countermeasure A.

For Countermeasure B, a plurality of detector boards (of which only 151 and 152 are shown) are used. A fluorescence filter is present on the fibers to filter out light that is not infrared (wavelengths below 780 nm). Because of the fluorescence filters, ambient light (with wavelengths below 780 nm) may not be detected. The fluorescence filter of the fiber 132 has been removed for detecting the ambient light. The detector controller 154 gets a command from the computer 155 to perform a functional measurement (imaging the mammalian body part for tumor detection). The detector controller signals the shutter 110 to open. Subsequently the detector controller 154 triggers the detector boards 151 and 152 to perform the functional measurement. The detector boards 151 and 152 pass the detected values to the detector controller 154. Then the detector controller 154 closes the shutter 110 and passes the detected values to the computer 155. This is for generating measured values to an external image processing system (not shown). At regular intervals, for example twice a second, interleaved with functional measurements, the detector controller 154 is commanded to do a measurement of the ambient light level in the holding device 130. For detecting the ambient light level in the holding device 130 the same steps take place but the shutter 110 is not opened during the ambient light level measurement. The light level in the holding device 130 is checked through the fibers 132 and 133. Failure is signaled through the signal HOLDING DEVICE_SAFE_B.

HOLDING DEVICE_SAFE_B=NO AMBIENT LIGHT IN THE HOLDING DEVICE=DARKNESS IN THE HOLDING DEVICE

In addition, the check is executed on the plurality of detector boards prior to the imaging of the mammalian body part. In this case the fluorescence filters are not active and also all the fibers are used to check the light level in the holding device 130. This is the Countermeasure B.

The two HOLDING DEVICE_SAFE signals are redundant. This is to ensure safety against “single component failure”.

The safety switch 157 is attached to the fiber switch 120 and generates a FS_SAFE signal. The safety circuit 156 controls the laser light source 100. The safety circuit 156 allows the laser light source to emit laser light and lets the laser light enter the holding device 130 only when the fiber switch 120 is stopping the beam or both the HOLDING DEVICE_SAFE signals are active.

LASER_ON=(FS_SAFE) OR (HOLDING DEVICE_SAFE_A AND HOLDING DEVICE_SAFE_B).

The computer 155 collects the signals from the detector boards 151 and 152 and transfers these signals to the image processing system. The computer 155 also determines the HOLDING DEVICE_SAFE_B signal. Thus, the computer serves to command the detector controller 154 to perform functional measurements and to collect the measured values from the detector controller 154.

According to another embodiment of the invention, a helper light may be installed to enhance the detection of light leakage from the holding device 130. In the previous embodiment, it is required to have the ambient light source 140 active all the time. On the other hand, the helper light (in the infrared range, so that it is not visible) can be activated only when the light level is checked in the holding device 130. During the functional measurement (i.e., when the laser light enters the holding device 130), the helper light would be off.

The light level in the holding device 130 is measured only in the intervals between the functional measurements. During the functional measurement, the safety checks are idle (a light level check in the holding device would fail because of the laser light). This idle period can be avoided by mounting filters in front of the dedicated detector 153 that detects the light level in the holding device 130. These filters block the laser light, but allow the ambient light to pass through. The laser light is near infrared. Therefore, these filters would need to block near infrared and infrared, and would need to pass light of a shorter wavelength. The ambient light would need to contain light of these shorter wavelengths, for instance green, blue or ultraviolet.

It is to be understood that although preferred embodiments, specific constructions and configurations have been discussed herein for the device according to the present invention, various changes or modifications in form and detail may be made without departing from the scope and spirit of this invention.

1. A diffuse optical tomography device comprising:
a light source for irradiating a mammalian body part;
a holding device configured for holding the body part;
a measuring device configured for measuring the light level in the holding device and the ambient light level; and
a controlling device configured for controlling the light source as a function of the measured light level in the holding device and the ambient light level.

2. The diffuse optical tomography device of claim 1, wherein the measuring device comprises:
a first series of optical fibers configured for sensing the light level in the holding device;
a second series of optical fibers configured for transmitting the ambient light level; and
a third series of optical fibers configured for transmitting scattered light from the holding device.

3. The diffuse optical tomography device of claim 1, wherein the controlling device comprises a shutter configured for preventing or allowing the light from the light source to enter into the holding device as a function of the light level in the holding device and the ambient light level.

4. The diffuse optical tomography device of claim 1, further comprising a coupling fluid disposed in the holding device to surround the body part, wherein the coupling fluid has an optical characteristic substantially identical to an optical characteristic of the body part.

5. The diffuse optical tomography device of claim 1, wherein the holding device includes a plurality of entrance openings for coupling light generated by the light source into the holding device and a plurality of exit openings constructed to communicate scattered light to the measuring-controlling device, the scattered light being emitted from the body part in response to light from the light source.

6. The diffuse optical tomography device of claim 1, wherein the light source is a laser light source.

7. A method for controlling a light source in a diffuse optical tomography device, comprising the steps of:
placing a mammalian body part in a holding device of a diffuse optical tomography device;
irradiating the body part in the holding device by means of a light source;
measuring the light level in the holding device by a measuring device;
measuring the ambient light level by the measuring device; and
controlling the light source, based on the measured light level in the holding device and the ambient light level.

8. The method of claim 7, wherein the light source is a laser light source.

9. A measuring device and a controlling device suitable for use in the tomography device according to claim 1, configured to measure the light level in the holding device and to measure the ambient light level and to control the light source of the tomography device as a function of the measured light level in the holding device and the ambient light level.
